(19) United States
(12) Patent Application Publication
(10) Pub. No.: US 2007/0275366 A1
(43) Pub. Date: Nov. 29, 2007

(54) Regulation of Sperm Function

(76) Inventor: Gavin Vinson, London (GB)

Correspondence Address: FISH & RICHARDSON PC, P.O. BOX 1022, MINNEAPOLIS, MN, 55440-1022, US

(21) Appl. No.: 10/580,081

(22) PCT Filed: Nov. 22, 2004
(86) PCT No.: PCT/GB04/04912

§ 371 Date: Feb. 14, 2007

(30) Foreign Application Priority Data
Nov. 20, 2003 (GB) 0327046.9

(51) Int. Cl. A01N 1/02; A61K 38/00; A61P 15/08

(52) U.S. Cl. 435/2; 514/2

(57) ABSTRACT

An extracellular matrix protein, such as fibronectin, vitronectin or laminin, is added to a sperm sample to bring the sperm into a low motility, non-capacitated state or maintain a pre-existing non-capacitated state. Subsequently, at an appropriate time in an in vitro fertilization study or in vivo fertilization procedure, angiotensin II or a related peptide is added to the sperm sample to enhance motility and capacitate the sperm. The extracellular matrix protein prevents undesirable early natural capacitation, and artificial insemination procedures can be made more precise, as sperm can be brought to the right state of capacitation for transfer at precisely the right time. The treatment with angiotensin II can be combined with use of the peptide RGD to compete with the binding of the extracellular matrix protein and so suppress its effect.

### FIELD OF THE INVENTION

This invention is concerned with the regulation of sperm function. In particular this invention relates to the use of specific proteins, such as fibronectin and angiotensin II, to respectively conserve sperm in a non-capacitated or non-activated state and to convert non-capacitated/inactivated sperm to the capacitated/activated state.

### BACKGROUND TO THE INVENTION

The physiological factors which induce and maintain mammalian sperm maturation and motility generally remain unclear, although several agents are known to be involved. For example, motility data on stimulated and unstimulated sperm from volunteers and patients attending fertility clinics showed that angiotensin II may increase both the percentage of motile sperm and their linear velocity, while the specific AT1 receptor antagonist losartan inhibits the action of angiotensin II on the percentage of motile sperm. It has been demonstrated that angiotensin II has actions on specific motility parameters, including curvilinear velocity, straight line velocity, and amplitude of lateral head movement. These motility changes are characteristically associated with sperm capacitation, that is the capacity, eventually, to fertilize ova. These findings are the basis for the invention of WO 95/32725 “Use of angiotensin II to promote fertility”.

At the same time, interest has been drawn to the interaction of sperm with extra-cellular matrix proteins, including particularly fibronectin. Fibronectin has been found in various locations on the surface of sperm, and its presence on sperm may be associated with infertility.

In other studies, the problems associated with sperm freezing have been discussed. Sperm freezing is an essential tool in sperm preservation for artificial insemination and in vitro fertilization in both domestic animals and in humans. However, depending on species and individual, there are varying degrees of damage that are associated with sperm freezing and thawing, and fertility is impaired compared with fresh unfrozen sperm. In part this is because freezing and thawing appears to elicit changes that reflect capacitation.

### SUMMARY OF THE INVENTION

This invention is based on the finding that extracellular matrix proteins such as fibronectin can be used as an additive to conserve sperm in a non-capacitated state by reducing motility, and that angiotensin II and its analogs and small peptides containing the RGD tripeptide can be used to capacitate samples by enhancing motility where capacitation has been suppressed by presence of an extracellular matrix protein.

Accordingly the present invention provides a sperm regulation method which comprises providing a sperm sample containing an extracellular matrix protein so that the sperm is in a non-capacitated state, and adding angiotensin II or a related peptide to capacitate the sperm.

Typically the sperm sample containing an extracellular matrix protein is prepared by adding an extracellular matrix protein to a sperm sample to bring into a non-capacitated state by reducing motility or maintain a pre-existing non-capacitated state. This use of matrix proteins to conserve sperm in a low motility, non-capacitated state is a further aspect of the invention. However the regulation method of the invention may also be used with sperm samples which naturally contain an extracellular matrix protein.

### BRIEF DESCRIPTION OF THE DRAWINGS

In the accompanying drawings:

FIG. 1 is a graphical representation of motility measurements from the procedures of Example 1;

FIG. 2 is a graphical representation of motility measurements from the procedures of Example 2;

FIG. 3 is a graphical representation of motility measurements from the procedures of Example 3;

FIG. 4 is a graphical representation of motility measurements from the procedures of Example 4;

FIG. 5 is a graphical representation of motility measurements from the procedures of Example 5; and

FIGS. 6, 7, 8 and 9 are graphical representations of capacitation measurements from the procedures of Example 6.

### DETAILED DESCRIPTION OF THE INVENTION

Sperm that are naturally in a non-capacitated state have the inherent potential eventually to proceed to capacitation, whether stimulated or not. However freezing and thawing sperm is quite damaging to them, but those that are recovered in viable condition after thawing, or after removing samples from refrigerated (but not frozen) storage, often proceed rapidly to capacitation, frequently limiting their usefulness post-thawing or post-chilling. A feature of the present invention is to make use of added extracellular matrix protein and angiotensin II to offer some control over the process, in effect providing a “brake and accelerator” under control of the technician using the sperm, for example in fertilisation studies or in treatment of patient samples in human fertility clinics or in artificial insemination of animals. By use of matrix proteins in accordance with this invention, it has been found to be possible to extend the usable life of sperm samples by 3-4 times.

In a preferred aspect, the present invention comprises adding an extracellular matrix protein to a sperm sample to bring the sperm into a non-capacitated state or maintain a pre-existing non-capacitated state, and subsequently, at an appropriate time in an in vitro fertilization study or in vivo fertilization procedure, adding angiotensin II or a related peptide to the sperm sample to capacitate the sperm.

In one aspect the present invention comprises the use of one or more extracellular matrix proteins as an agent to conserve sperm in a non-capacitated/inactive state by reducing motility. Effective amounts can be found by routine testing using the Examples below for guidance. For example matrix proteins may be added to samples at levels of about 1 μg/ml to 50 μg/ml, typically from 2 μg/ml to 20 μg/ml.

The extracellular matrix protein may be added to fresh sperm, suitably in conventional extender media, for cold storage or before freezing for cryo-preservation as frozen sperm.

Alternatively the extracellular matrix protein may be added to sperm samples post-thawing or post-chill storage, when the samples are being held ready for use. Thawed samples are typically suspended in a proprietary medium by the supplier. The technician responsible for IVF procedures or conducting fertilisation studies may find it convenient to wash the thawed sperm, concentrate the spermatozoa by centrifugation, and then re-suspend the sperm in a medium suitable for the studies or IVF.

In another aspect the present invention comprises the use of angiotensin II or related peptides as an agent for capacitation of sperm samples by enhancing motility when the samples have been conserved in a low motility, non-capacitated state by the presence of an extracellular matrix protein. Effective amounts can be found by routine testing using the Examples below for guidance. For example it may be suitable to provide the angiotensin II or analog at amounts of 0.1 nM to 100 nM, typically 1 nM to 10 nM, in the treated composition.

The extracellular matrix protein-containing sperm samples that provide non-capacitated sperm may result from thawing extracellular matrix protein-containing frozen sperm, or from extracellular matrix protein-containing sperm samples that have been stored unfrozen, or from sperm samples that naturally contain a extracellular matrix protein.

Alternatively the extracellular matrix protein may be added to sperm samples post-thawing or post-storage, or to fresh samples, followed by use of angiotensin II or related peptide, so that a technician can hold the sperm in a low motility non-capacitated state until needed for use, when capacitation is induced using the angiotensin II.

The extracellular matrix proteins are capable of binding to cell surfaces and this is believed to be involved in maintaining sperm in a low motility, non-capacitated state in accordance with this invention, although the actual mechanism is not yet known. Suitable extracellular matrix proteins for use in the present invention include fibronectin, vitronectin and laminin.

As a capacitating agent, angiotensin II can be replaced by related peptides such as the salts and analogs mentioned in WO 95/32725, the entire disclosure of which is incorporated herein by reference. A suitable analog is angiotensin II amide.

Alternatively peptides containing the tripeptide motif RGD (Arg-Gly-Asp) may be used as agents for capacitation. This motif may be provided by the tripeptide RGD itself or by other small peptides, such as the commercially available tetrapeptide RGDS (Arg-Gly-Asp-Ser). RGD is the tripeptide motif found in all the extracellular matrix proteins, to which the integrins in the cell membrane bind. Use of free RGD, or other peptides containing the motif RGD, therefore competes with the extracellular matrix proteins, and will inhibit cell binding by the proteins. Effective amounts can be found by routine testing using the Examples below for guidance.

RGD is suitably used in combination with angiotensin II, because the RGD inhibits extracellular matrix protein binding, increasing the effectiveness of added angiotensin II in stimulating motility and hence capacitation/activation. However angiotensin II is still an effective capacitation agent in the absence of RGD peptides.

For storage, fresh sperm is preferably added to a sperm extender medium or other reproductive cell medium.

Accordingly a further aspect of the invention is a sperm extender medium containing an extracellular matrix protein such as fibronectin, vitronectin or laminin, so that added sperm is maintained in a non-capacitated state. Sperm extender media are commercially available and typically comprise a buffered aqueous solution.

A sperm extender medium is frequently prepared at the time of use by adding a solid, typically powdered, extended composition to water. Accordingly the present invention also provides a sperm extender composition, typically powder for reconstitution with water, containing an extracellular matrix protein.

In a still further aspect, the present invention provides use of one or more extracellular matrix proteins for the preparation of an agent for conserving sperm in a non-capacitated state or for preparation of a sperm extender medium for conserving sperm in a non-capacitated state.

When the extended medium is used for cryo-preservation of sperm, the extender medium also preferably also contains one or more cryoprotective agents such as glycerol.

Similarly, for capacitating sperm, a sperm sample may be added to an extender medium containing a capacitation agent or vice versa.

Accordingly the present invention also provides a sperm extender medium or composition containing angiotensin II or analog and/or a RGD-containing peptide.

In a still further aspect the present invention provides use of angiotensin II or analog and/or a RGD-containing peptide for the preparation of an agent for capacitating sperm or for preparation of a sperm extender medium or composition for capacitating sperm.

Sperm extended compositions are well know to the skilled person working in the field of this invention, and generally contain buffers, for example citric acid or sodium citrate and sodium bicarbonate, and/or nutrients, for example sugars such as glucose. When the sperm is to be stored frozen then, as is also well known, cryo-protectants such as glycerol or egg or milk proteins may be added. Antibiotics may also be added. The pH of the medium is generally about 7-8.

In general, the procedures of this invention will make use of media for reproductive cells which are commercially available or well known in the art—see for example the materials listed in WO 03/072707, the entire disclosure of which is incorporated herein by reference.

This invention is primarily concerned with mammalian sperm, especially human sperm for use in fertility clinics and animal sperm for use in stock-breeding by artificial insemination.

A major problem with both human and domestic animal artificial insemination procedures is the timing of sperm capacitation to ovulation. Partly because of this, success rates are often very low. By using “brake and accelerator” agents in accordance with the present invention, artificial insemination procedures can be made more precise. In a suitable extender medium, sperm can be transferred at the right state of capacitation at precisely the right time. In some circumstances, the use of a extracellular matrix protein “brake” may be a sufficient intervention, and the sperm can be capacitated by the natural release of angiotensin II that accompanies ovulation. This extends the application of the invention, by avoiding therapeutic intervention for timing of ovulation.

A further development of the invention provides both sperm enhancement and inhibition (fertility enhancement and contraception) in vivo by the use of internally applied pessaries, containing extracellular matrix protein (e.g. fibronectin) or angiotensin II (or analog) shortly before sexual intercourse.

Accordingly the invention provides in a further aspect provides:

a sperm inhibition composition comprising one or more extracellular matrix proteins, preferably dispersed in a pessary base;

a sperm enhancement composition comprising angiotensin II or a related peptide, preferably dispersed in a pessary base.

Suitable pessary bases for human and animal use are commercially available and well known in the art. Typically they comprise fats or waxes that melt at appropriate temperature for internal use. Foam pessaries are also usable to assist in dispersion of the active substance. Creams and gels may also be used as vehicles.

The invention is illustrated, by way of example only, in the following Examples.

Materials

Bull semen samples (Semex)

Fibronectin (250 μg/ml, Sigma)

Vitronectin (250 μg/ml, Sigma)

Laminin (250 μg/ml, Sigma)

RGDS (150 μl/ml) (as a source of RGD)

Angiotensin II (10⁻⁹ moles/L)

Sp-TALP (Tyrode's Albumin Lactate Pyruvate) Media (TL, BSA, Fract V, Pyruvate, gentamicin)—a reproductive cell medium

Thermoplate—a heated microscope stage

Sperm Preparation and Separation:

Each sperm sample was packed and frozen in 0.25 ml plastic straws. The frozen spermatozoa straw (0.25 ml) was rapidly thawed by plunging it into a 37° C. water bath. When the sample was thawed, the straw was dried with a tissue, kept horizontal and cut both ends into a pre-warmed TALP medium.

Capacitation

Spermatozoa were washed twice by TALP medium and centrifugation at 600×g for 10 minutes, then re-suspended with TALP pre-warmed to 37° C. The motility of the frozen-thawed spermatozoa was assessed at 37° C. using thermal plates. Sperm motility and morphology was estimated by microscopical observation on a warm 37° C. stage. Semen (10 μl) was covered by a cover glass (18 mm×18 mm) and at least 5 microscopic fields were examined. Motility was expressed as the percentage of motile spermatozoa with linear progressive movement in the sample (see Vinson, G. P., Puddefoot, J. R., Ho, M. M., Barker, S., Mehta, J., Saridogan, E. and Djahanbakhch, O. (1995). “Type 1 angiotensin II (AT1) receptors in sperm”, Journal Of Endocrinology, 144, 369-378). The prepared sperm sample were incubated with additives at 37° C. in a water bath as described in each example, and assayed for motility as before.

### EXAMPLE 1

Samples of thawed sperm were incubated with additives as follows:

1. Sperm were incubated with fibronectin (2 μg/l) for 10 minutes.

2. Sperm were incubated with fibronectin (2 μg/l) plus RGDS (5×10⁻⁶ moles/L) for 10 minutes.

3. Angiotensin II (10⁻⁹ moles/L) was added to the fibronectin plus RGDS sample and incubated for further periods of 10, 20, 30 and 40 minutes.

All incubations were carried out at 37° C., using water bath and heated stage. Semen samples (10 μl) were applied to preheated (37° C.) slides and placed on the microscope heated stage and examined. The number of motile sperm were counted in the control sample at zero time, and in the treated samples after the incubation times indicated.

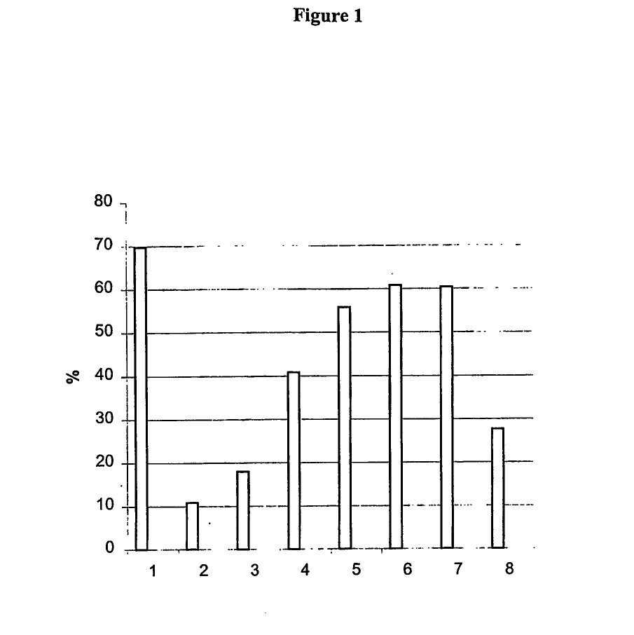

The results are shown in FIG. 1 in which the columns show motility values for

1. control 0 time;

2. fibronectin 10 min;

3. fibronectin+RGD 10 min;

4. fibronectin+RGD+Angiotensin II 10 min;

5. fibronectin+RGD+Angiotensin 1120 min;

6. fibronectin+RGD+Angiotensin II 30 min;

7. fibronectin+RGD+Angiotensin II 40 min;

8. control after 40 min;

from which it can be seen that:

a) Control sample showed 70% motility at the time zero

b) The effects of fibronectin concentration on the sperm showed a highly significant change in total sperm motility. After incubation of 5-10 minutes the sample showed only 14% sperm motility.

c) The effects of fibronectin plus RGDS after 10 minutes showed no significant difference (23%) when compared to fibronectin alone for 10 minutes.

d) By addition of Angiotensin II to the sample, there was a significant change in sperm motility. Increased incubation with Angiotensin II showed an increase in sperm motility. After 10, 20, and 3040 minutes incubation the motility was increased from 43%, 64% and 71% respectively compared to fibronectin alone. Compared to the RGD plus fibronectin sample the increase was 34%, 55%, and 62% respectively.

e) The control sample showed only 28% motility after 40 minutes incubation in the water bath.

### EXAMPLE 2

Samples of thawed sperm were incubated with additives as follows:

1. Sperm were incubated with fibronectin (2 μg/l) for 10 min.

2. Angiotensin II (10⁻⁹ moles/L) was added to the fibronectin sample and incubated for further periods of 10, 20, and 30 minutes.

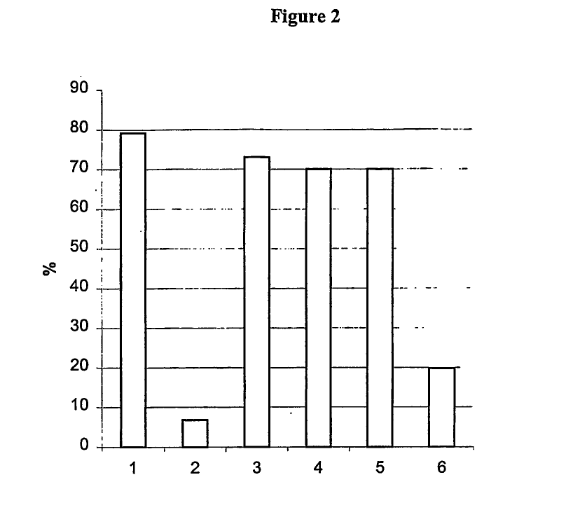

The results are shown in FIG. 2 in which the columns show motility values for

1) control 0 min;

2) fibronectin 10 min;

3) fibronectin+Angiotensin II 10 min;

4) fibronectin+Angiotensin II 20 min;

5) fibronectin+Angiotensin II 30 min;

6) control after 30 min;

from which it can be seen that:

a) Control sample showed 79% motility at the time zero

b) The effects of fibronectin concentration on the sperm showed a highly significant change in total sperm motility. After incubation of 5-10 minutes there was only 8% sperm motility.

b) By addition of Angiotensin II to the sample, there was a significant change in sperm motility. At the time 10, 20, and 3040 minutes from the start of incubation the motility was increased to 64%, 62% and 62% respectively compared to fibronectin alone.

c) The control sample showed only 20% motility after 30 minutes incubation in the water bath.

### EXAMPLE 3

Samples of thawed sperm were incubated with additives as follows:

1. Sperm were incubated with RGDS (5×10⁶ moles/L) for 5 min.

2. Fibronectin (2 μg/l) was added to the RGDS sample and incubated for a further 5 min.

3. Angiotensin II (10⁻⁹ moles/L) was added to the fibronectin+RGDS sample and incubated for further periods of 10, 20, and 30 minutes.

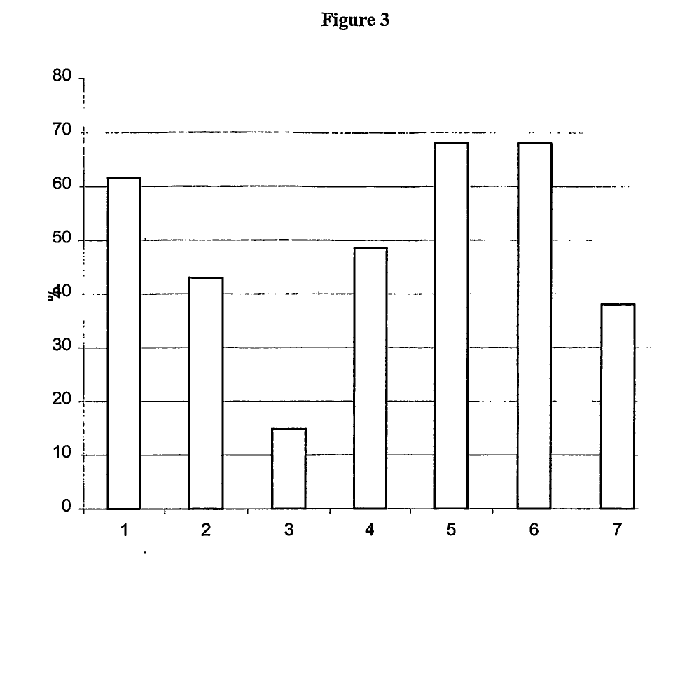

The results are shown in FIG. 3 in which the columns show motility values for

1) control 0 min;

2) pre-incubation with RGD 5 min;

3) RGD+fibronectin 5 min;

4) fibronectin+RGD+Angiotensin II 10 min;

5) fibronectin+RGD+Angiotensin II 20 min;

6) fibronectin+RGD+Angiotensin II 30 min;

7) control 30 min;

from which it can be seen that:

a) Control sample showed 62% motility at the time zero.

b) Pre-incubation of RGDS with sperm showed 19% decrease in sperm motility.

c) The effects of fibronectin concentration on the pre-incubated RGDS with sperm showed a highly significant change in total sperm motility. After incubation of 5-10 minutes there was a 47% and 28% decrease of motility compared to the control and RGD treated samples respectively.

d) By addition of Angiotensin II to the sample, there was a significant change in sperm motility. The longer the incubation with Angiotensin II showed an increase in sperm motility. After 10, 20, and 3040 minutes incubation the motility was increased from 43%, 54% and 54% respectively compared to 20% fibronectin and RGDS.

e) The control sample showed only 38% motility after 30 minutes incubation in the water bath.

### REPETITION OF EXAMPLES 1 TO 3

The procedures of Examples 1 to 3 were carried out using the matrix protein fibronectin at 2 μg/l. Carrying out the same procedures with 20 μg/l fibronectin achieved similar results.

### EXAMPLE 4

Samples of thawed sperm were incubated with additives as follows:

1. Sperm were incubated with vitronectin (2 μg/l) at 37° C. in water bath for 5 minutes.

2. Angiotensin II (10⁻⁹ moles/L) was added to the vitronectin sample and incubated for further periods of 5, 15, 15 and 35 minutes.

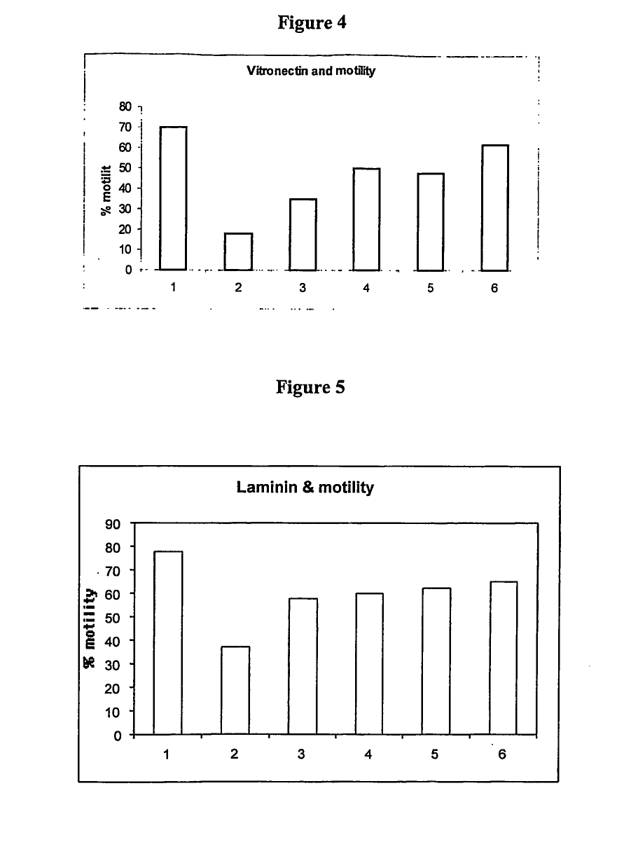

The results are shown in FIG. 4 in which the columns show motility values for

1) control 0 min;

2) vitronectin 5 min;

3) vitronectin+Angiotensin II 5 min;

4) vitronectin+Angiotensin II 15 min;

5) vitronectin+Angiotensin II 15 min;

6) vitronectin+Angiotensin II 35 min;

from which it can be seen that:

the effect of vitronectin concentration on the sperm showed a highly significant decrease in total sperm motility; and by addition of Angiotensin II to the sample, there was a significant increase in sperm motility.

### EXAMPLE 5

Samples of thawed sperm were incubated with additives as follows:

1. Sperm were incubated with laminin (2 μg/l) at 37° C. in water bath for 10 min.

2. Angiotensin II (10⁻⁹ moles/L) was added to the laminin sample and incubated for further periods of 10, 20, 30 and 40 minutes.

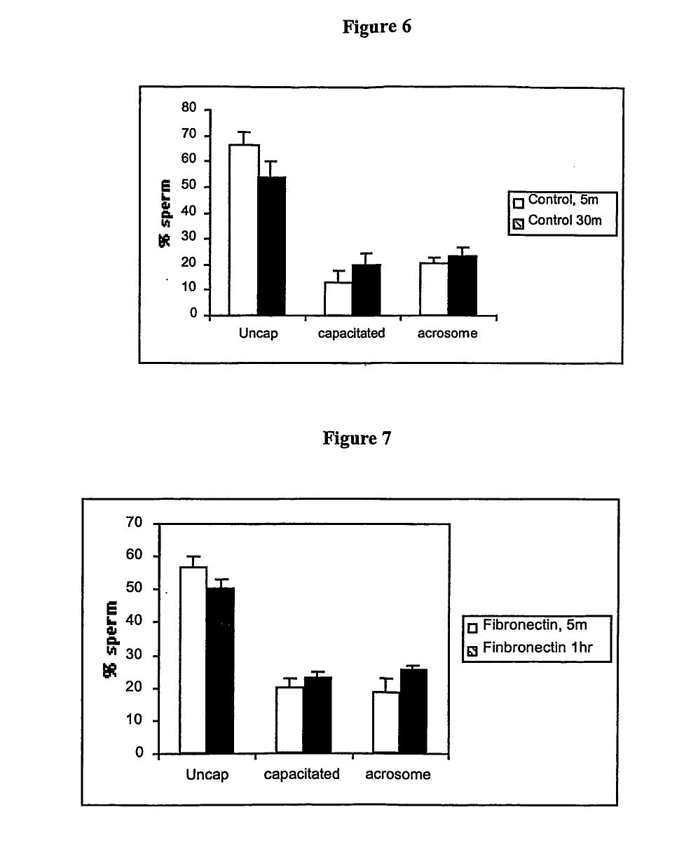

The results are shown in FIG. 5 in which the columns show motility values for

1) control 0 min;

2) laminin 5 min;

3) laminin+Angiotensin II 10 min;

4) laminin+Angiotensin II 20 min;

5) laminin+Angiotensin II 30 min;

6) laminin+Angiotensin II 40 min;

from which it can be seen that the effects of laminin concentration on the sperm showed a significant decrease in total sperm motility; by addition of Angiotensin II to the sample, there was a significant increase in sperm motility.

Motility and Capacitation

Capacitation, at basis, is the capacity of sperm to fertilise ova. Immotile sperm cannot fertilise ova, and usually the initiation of hyperactivation is thought to be coincident with capacitation. The data in the above Examples all show percentage motility. The stimulating effect of angiotensin II increases the motility of sperm and also the curvilinear velocity, lateral head displacement, and beat cross frequency associated with hyperactivation (Vinson, G P, Mehta, J, Evans, S, Matthews, S, Puddefoot, J R, Saridogan, E, Holt, W V, and Djahanbakhch, O (1996) Angiotensin ii stimulates sperm motility. Regulatory Peptides 67 131-135.)

An alternative method of assessing capacitation is by using the CTC assay (DasGupta S, Mills C L, Fraser L R. Ca(2+)-related changes in the capacitation state of human spermatozoa assessed by a chlortetracycline fluorescence assay. J Reprod Fertil. 1993 September; 99(1):135-43.; Fraser L R, Herod J E. Expression of capacitation-dependent changes in chlortetracycline fluorescence patterns in mouse spermatozoa requires a suitable glycolysable substrate. J Reprod Fertil. 1990 March; 88(2):611-21). In this method, used in Example 6 below, patterns of staining distinguish non-capacitated, capacitated and acrosome reacted sperm.

### EXAMPLE 6

Materials:

Chlortetracycline (CTC) solution: CTC (750 μM CTC in Tris (20 mM)-NaCl (130 mM), DL-cysteine (5 mM), buffer (20 mM Tris/130 mM NaCl, pH 7.8).

Angiotensin II (10⁻⁹) moles per litre

Fibronectin (5-50 μg/ml)

Spermatozoa samples were obtained from healthy bulls (Semex, UK). All semen samples used in this study had been frozen and thawed. Each sample was packed in 0.25 ml plastic straws.

Sperm Preparation and Separation:

The frozen spermatozoa straw (0.25 ml) was rapidly thawed by plunging it into a 37° C. water bath. When the sample was thawed, the straw was dried with a tissue, kept horizontal and cut both ends into a pre-warmed TALP medium.

Capacitation

Spermatozoa were washed twice by TALP medium and centrifugation at 600 g for 10 minutes, then re-suspended with TALP pre-warmed to 37° C. Spermatozoa were incubated without additives, or with either angiotensin II (10⁻⁹ moles per litre), fibronectin (5-50 μg/ml), or with both. Treated samples (45 μl) were aliquoted into foil wrapped eppendorf tubes with CTC solution (45 μl) and fixed with 12.5% paraformaldehyde, prior to viewing by light microscopy.

Three patterns of chlortetracycline fluorescent staining may be observed on bull spermatozoa:

F (uncapacitated)-pattern, bright fluorescence was detected over the whole region of the sperm head; B (capacitated)-pattern, fluorescence was detected on the sperm head but not on the postacrosomal region; AR (acrosome-reacted)-pattern, weak fluorescence was observed over the sperm head, and a bright band was sometimes observed in the equatorial region.

Results

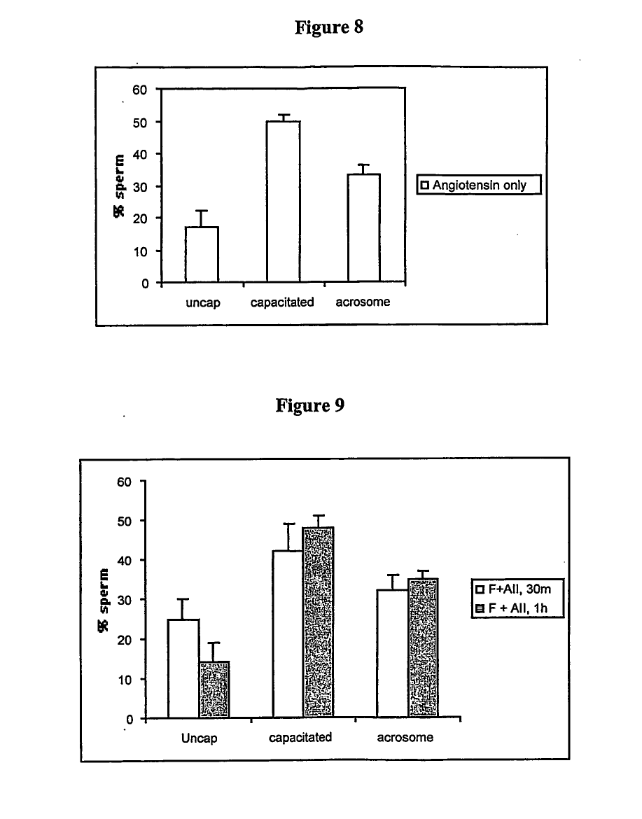

In the control (untreated) group about 60% of the sperm exhibited the uncapacitated pattern and by 30 min this had fallen to 55%. There were no significant changes in capacitated and acrosome reacted sperm (see FIG. 6).

Fibronectin (5 μg/ml) treated samples showed patterns of sperm staining very similar to the untreated controls (compare FIG. 6 and FIG. 7).

In contrast, 60 min incubation with angiotensin II only, there was an increase in capacitated sperm (45%) and uncapacitated sperm fell to about (18%) (see FIG. 8). These data suggest that angiotensin II promotes the uncapacited (F pattern) to capacitated (B pattern) transition. This is followed by rapid acrosome exocytosis. There was also a marked decrease in uncapacitated sperm in the samples treated with both fibronectin (5 μg/ml) and angiotensin II (10⁻⁹), and within 30 min, only 25% of cells exhibited the uncapacitated pattern and by 60 min this had fallen to 15%. This was accompanied by increases in capacited and acrosome reacted sperm throughout (see FIG. 9).

The results show that Ang II (10⁻⁹) will increase capacitation, even in the presence of fibronectin, and that, from comparison with the data given in previous Examples, angiotensin II stimulation of motility and capacitation are synchronous.

### EXAMPLE 7

### Action of Fibronectin on Sperm Viability

Stock solutions: carboxyfluorescein diacetate (0.46 mg/ml in dimethyl sulphoxide), propidium iodide (0.5 mg/ml isotonic saline), formaldehyde (10 μl formalin to 150 μl distilled water). Fluorescence stain: 50 μl carboxyfluorescein and 50 μl of propidium iodide to 2 ml sperm extender, plus 100 μl formaldehyde.

Semen were diluted with stain (1:1) dilution and incubated at room temperature at 37° C. for 15 min. Samples washed with extender, and examined for live and dead sperm.

In untreated control samples, showed that percentages of living sperm decreased from 75% to 18% when stored at room temperature for three days. Sperm kept in identical conditions, but in the additional presence of fibronectin (5 μg/ml), showed 48% remained viable after 3 days.

1-19. (canceled)

20. A sperm regulation method which comprises adding an extracellular matrix protein to a sperm sample to bring the sperm into a non-capacitated state, and subsequently adding angiotensin II or a salt or analog to stimulate motility and capacitate the sperm.

21. A method according to claim 20, in which the sperm sample containing an extracellular matrix protein has been stored prior to adding angiotensin II or a salt or analog to capacitate the sperm.

22. A method according to claim 21 in which the sperm sample containing an extracellular matrix protein has been frozen or chilled for storage and is thawed prior to adding angiotensin II or a salt or analog to capacitate the sperm.

23. A method according to claim 20 which is carried out in vitro.

24. A method according to claim 20 in which addition of angiotensin II or a salt or analog is carried out in vivo.

25. A method according to claim 20 which is carried out in vivo.

26. A method according to claim 20 in which the extracellular matrix protein is selected from fibronectin, vitronectin and laminin.

27. A method according to claim 20 in which angiotensin II or angiotensin II amide is used as the capacitating agent

28. A sperm conservation method which comprises adding an extracellular matrix protein to a sperm sample to bring the sperm into a non-capacitated state,

29. A method according to claim 28 in which the extracellular matrix protein is selected from fibronectin, vitronectin and laminin.

30. A sperm conservation method which comprises adding an extracellular matrix protein to a sperm sample to conserve the sperm in a low motility non-capacitated state.

31. A method according to claim 30 in which the extracellular matrix protein is selected from fibronectin, vitronectin and laminin.

32. A reproduction cell medium comprising one or more extracellular matrix proteins dispersed in a buffer solution, for use as an agent to conserve sperm in a non-capacitated state.

33. A reproduction cell medium according to claim 32 in which the extracellular matrix protein is selected from fibronectin, vitronectin and laminin.

34. A sperm inhibition composition comprising one or more extracellular matrix proteins dispersed in a gel, cream or pessary base, for use as an agent to inhibit capacitation of sperm.

35. A reproduction cell medium according to claim 34 in which the extracellular matrix protein is selected from fibronectin, vitronectin and laminin.
